(19) United States
(12) Patent Application Publication
(10) Pub. No.: US 2025/0118406 A1
(43) Pub. Date: Apr. 10, 2025

(54) OPTIMIZING DRUG COMBINATIONS FOR TREATING ACUTE HEART FAILURE

(71) Applicant: NTT RESEARCH, INC., Sunnyvale, CA (US)

(72) Inventors: Yasuyuki KATAOKA, Sunnyvale, CA (US); Yukiko FUKUDA, Sunnyvale, CA (US); Jon PETERSON, Sunnyvale, CA (US)

(73) Assignee: NTT RESEARCH, INC., Sunnyvale, CA (US)

(21) Appl. No.: 18/868,013

(22) PCT Filed: May 25, 2023
(86) PCT No.: PCT/US2023/067510

§ 371 Date: Nov. 21, 2024

Related U.S. Application Data
(60) Provisional application No. 63/346,143, filed on May 26, 2022.

(51) Int. Cl. G16H 20/10; A61B 5/00; G16H 10/60

(52) U.S. Cl. CPC G16H 20/10; A61B 5/4848; G16H 10/60

(57) ABSTRACT

Embodiments disclosed herein may include operations of receiving a plurality of current cardiovascular performance metrics of a patient and a plurality of candidate drugs to be used to reach a plurality of desired cardiovascular performance metrics and determining optimal dosages of the plurality of candidate drugs to reach the plurality of desired cardiovascular performance metrics. The determining may include optimizing a dosage combination of the plurality of candidate drugs to reach a plurality of desired cardiovascular parameters, corresponding to the plurality of desired cardiovascular performance metrics, from a plurality of current cardiovascular parameters corresponding to the plurality of current cardiovascular performance metrics and mapping the plurality of desired cardiovascular performance metrics from the plurality of desired cardiovascular parameters and the plurality' of current cardiovascular performance metrics from the plurality of current cardiovascular parameters. The operations may further include outputting

### CROSS REFERENCE TO RELATED APPLICATIONS

This application claims priority from U.S. Provisional Application No. 63/346,143, filed May 26, 2022, and entitled “Optimizing Drug Combinations for Treating Acute Heart Failure,” which has been incorporated by reference in its entirety.

### FIELD

This disclosure relates to determining an optimal combination of drugs for treating acute heart failures, particularly to linearly optimizing a drug combination to achieve, for acute heart failure patients, desired cardiovascular parameters that map to desired cardiovascular performance metrics.

### BACKGROUND

Acute heart failure is caused by different factors and therefore generally requires complex drug therapies with multiple drugs (also referred to as medications). Some examples of the drugs for treating acute heart failure include positive inotropes such as dobutamine, vasopressors such as norepinephrine, vasodilators such as sodium nitroprusside, fluids such as dextran, diuretics such as furosemide, etc. Each of these drugs may treat a different, specialized aspect of acute heart failure. For more effective treatment of acute heart failure, an optimal combination of these drugs is desired. An example optimal combination may include a minimum amount of drug that treats a corresponding condition while minimizing the side effects.

Finding such an optimal combination, however, involves several technical challenges. The pharmacologic effect of each of the drugs in the combination is by itself complex and the complexity quickly increases when multiple drugs are involved. One challenge is to understand and leverage complex dependencies/causalities between the drugs, cardiovascular parameters, and cardiovascular performance metrics. Another challenge is to find an optimal combination within the constraints of dosage limitation while considering unclear interactions between the different dosages.

Technical solutions have been devised to address these technical issues, but these solutions remain unsatisfactory. Generally, conventional technical solutions are based on single input single output (SISO) paradigm of how a drug drives an outcome based on a patient's pathophysiology. One study, for example, evaluates effect of norepinephrine on mean arterial pressure. But SISO is just for a single drug for driving single outcome type, and by design, does not address a multiple drugs scenario—let alone an optimal combination of the multiple drugs. Multiple SISO control studies have also been performed, but these studies have failed to consider—and failed to evaluate—unknown interactions between the different SISO systems.

Therefore, clinical decision making for an optimal combination of drug is therefore driven by trial and error and guesswork; and is inherently inaccurate. Such undesirable situation creates unnecessary hardships on the patients. The health outcomes are less than desirable and mortality rate among heart failure patients remains unnecessarily high. As such, a significant improvement in systems, methods, and devices to assist clinical decision-making for an optimal combination of drugs in acute heart failure patients is therefore desired.

### SUMMARY

In some embodiments, a computer readable non-transitory storage medium storing computer program instructions is provided. The computer program instructions when executed may cause operations that may include receiving a plurality of current cardiovascular performance metrics of a patient and a plurality of candidate drugs to be used to reach a plurality of desired cardiovascular performance metrics and determining optimal dosages of the plurality of candidate drugs to reach the plurality of desired cardiovascular performance metrics. The determining may include optimizing a dosage combination of the plurality of candidate drugs to reach a plurality of desired cardiovascular parameters, corresponding to the plurality of desired cardiovascular performance metrics, from a plurality of current cardiovascular parameters corresponding to the plurality of current cardiovascular performance metrics and mapping the plurality of desired cardiovascular performance metrics from the plurality of desired cardiovascular parameters and the plurality of current cardiovascular performance metrics from the plurality of current cardiovascular parameters. The operations may further include outputting the optimal dosages of the plurality of candidate drugs.

In some embodiments, a computer implemented method is provided. The method may include receiving a plurality of current cardiovascular performance metrics of a patient and a plurality of candidate drugs to be used to reach a plurality of desired cardiovascular performance metrics and determining optimal dosages of the plurality of candidate drugs to reach the plurality of desired cardiovascular performance metrics. The determining may optimizing a dosage combination of the plurality of candidate drugs to reach a plurality of desired cardiovascular parameters, corresponding to the plurality of desired cardiovascular performance metrics, from a plurality of current cardiovascular parameters corresponding to the plurality of current cardiovascular performance metrics and mapping the plurality of desired cardiovascular performance metrics from the plurality of desired cardiovascular parameters and the plurality of current cardiovascular performance metrics from the plurality of current cardiovascular parameters. The method may further include outputting the optimal dosages of the plurality of candidate drugs.

In some embodiments, a system is provided. The system may include a non-transitory computer readable medium storing computer program instructions and one or more processors configured to execute the computer program instructions to cause operations. The operations may include receiving a plurality of current cardiovascular performance metrics of a patient and a plurality of candidate drugs to be used to reach a plurality of desired cardiovascular performance metrics and determining optimal dosages of the plurality of candidate drugs to reach the plurality of desired cardiovascular performance metrics. The determining may include optimizing a dosage combination of the plurality of candidate drugs to reach a plurality of desired cardiovascular parameters, corresponding to the plurality of desired cardiovascular performance metrics, from a plurality of current cardiovascular parameters corresponding to the plurality of current cardiovascular performance metrics and mapping the plurality of desired cardiovascular performance metrics from the plurality of desired cardiovascular parameters and the plurality of current cardiovascular performance metrics from the plurality of current cardiovascular parameters. The operations may further include outputting the optimal dosages of the plurality of candidate drugs.

### BRIEF DESCRIPTION OF DRAWINGS

FIG. 1 depicts an example computing environment for assisting clinical decision-making in drug therapy for acute heart failure patients, according to example embodiments of this disclosure.

FIG. 2 shows details of a drug infusion module and a mapping module in the computing environment of FIG. 1, according to example embodiments of this disclosure.

FIG. 3 shows an example solution system of the analytical solution followed by the numerical solution of a mapping function utilized by the mapping module, according to example embodiments of this disclosure.

FIG. 4 shows an example solution system of numerical solution from input cardiovascular parameters to output cardiovascular performance metrics utilized by the mapping module, according to example embodiments of this disclosure.

FIG. 5 depicts a flow diagram of an example method, based on the example embodiments of this disclosure.

FIG. 6 shows an example system for treatability simulation, according to example embodiments of this disclosure.

FIG. 7 shows example graphs for a pathophysiological scenario analysis, according to example embodiments of this disclosure.

FIG. 8 shows a block diagram of an example computing device that implements various features and processes, according to example embodiments of this disclosure.

The figures are for purposes of illustrating example embodiments, but it is understood that the present disclosure is not limited to the arrangements and instrumentality shown in the drawings. In the figures, identical reference numbers identify at least generally similar elements.

### DESCRIPTION

Embodiments described herein solve the technical problems described above and may provide other solutions as well. An example optimal drug combination is calculated based on a decomposing a cardiovascular system to a first model with cardiovascular parameters affected by the drug combination and a second model with a mapping between the cardiovascular parameters and cardiovascular performance metrics. The first model, along with maximum dosage levels, provides the constraints for linearly optimizing the drugs combination; and the second model provides a mapping between cardiovascular parameters and cardiovascular performance metrics. The mapping may therefore determine whether the patient is treatable using the drugs combination, e.g., when the combination leads to cardiovascular performance metrics outside of a target area.

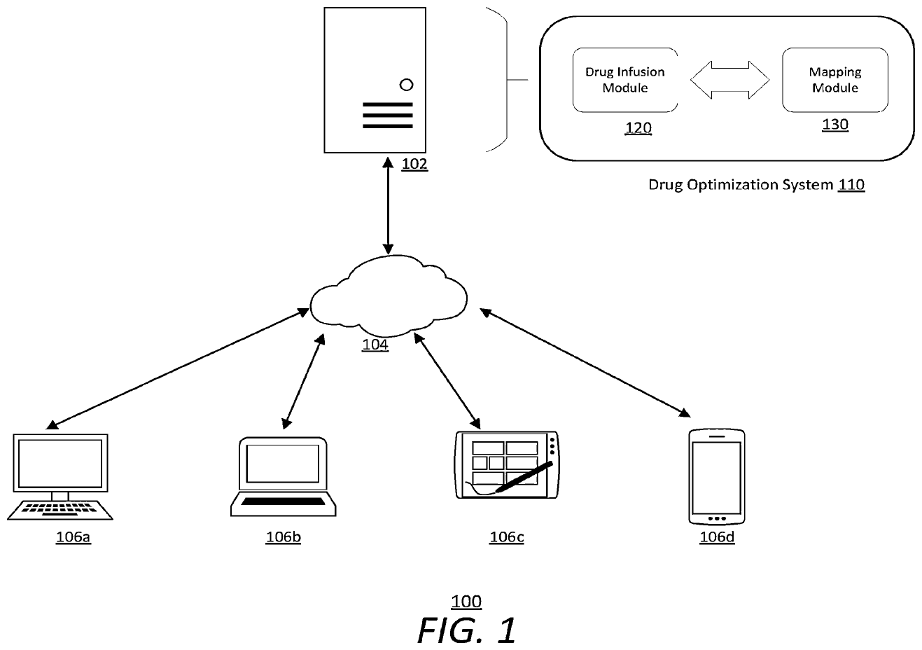

FIG. 1 depicts an example computing environment 100 for assisting clinical decision-making in drug therapy for acute heart failure patients, according to example embodiments of this disclosure. As shown, the computing environment 100 may be based on a client-server model, with a server 102 connected to multiple clients 106a-106d (commonly referred to as a client 106 or collectively referred to as clients 106) via a network 104. It should, however, be understood that the client-server model is just for illustration and ease of explanation and should not be considered limiting. Therefore, any type of computing environment performing the functionality disclosed herein should be considered within the scope of this disclosure. Furthermore, the individual components of the computing environment 100 are just illustrative and computing environments with alternative, additional, or fewer number of components should be considered within the scope of this disclosure.

The computing environment 100 may be generally in a clinical setting to assess clinical decision making for acute heart failure patients. In some example use cases, the server 102 may store different software modules that may be accessed by the clients 106 using the network 104. The clients 106 themselves may have standalone applications (not shown) to access the software modules. Alternatively, the clients 106 may access the software modules through a browser application, for example.

The hardware of the server 102 may be representative any kind of computing device. For example, the server 102 may include any kind of computing device, including but not limited to a server computer, a desktop computer, a laptop computer, a tablet computer, a smartphone. The server 102 may not necessarily be at a single location and may be realized by a network of computers. Furthermore, the server 102 may not necessarily be co-located within the clinical setting itself, and may be hosted by a third party cloud computing provider. Therefore, any kind of server 102 should be considered within the scope of this disclosure.

As described above, the clients 106 may access the server 102 through the network 104. The network 104 may include any combination of one or more packet switching networks (e.g., an IP based network) and one or more circuit switching networks (e.g., a cellular telephony network). Some non-limiting examples of the network 104 include a local area network, a metropolitan area network, a wide area network such as the Internet, etc. Similarly, non-limiting examples of the clients 106 may include a desktop terminal (e.g., desktop terminal 106a), a laptop computer (e.g., a laptop computer 106b), a tablet computer (e.g., a tablet computer 106c), a smartphone (e.g., a smartphone 106d), etc. Any type of computing device that allows an access to the server 102 through the network 104 should be considered within the scope of this disclosure. Furthermore, the functionality described within this disclosure can be distributed in any fashion, i.e., functionality of the server 102 may be performed by one or more clients 106 and vice versa.

The server 102 may include a drug optimization system 110 that may be configured to generate an optimal drug dosage for a patient based on received cardiovascular performance metrics data of the patient. For example, drug optimization system 110 may generate an optimal dosage of drugs to prescribe to the patient to achieve desired cardiovascular performance metrics based on a current measurement of cardiovascular performance metrics.

In some embodiments, the drug optimization system 110 may include one or more modules to generate a recommended combination of drugs to reach the desired cardiovascular performance metrics from current cardiovascular performance metrics. The drug optimization system 110 may include a drug infusion module 120 and a mapping module 130. As described in detail below, the drug infusion module 120 may be configured to deploy a first analytical model representing a transition from current cardiovascular parameters (corresponding to the current cardiovascular performance metrics) to a desired cardiovascular parameters (corresponding to the desired cardiovascular performance metrics). The mapping module 130 may be configured to deploy a second analytical model that may provide a mapping between the current set of cardiovascular parameters and the current set of cardiovascular performance metrics, and also the mapping between the target set of cardiovascular parameters and the target set of cardiovascular performance metrics.

After one or more analytical models have been developed and validated, clinicians may use the drug optimization system 110 to aid their clinical decision-making process for acute heart failure patients. In some example operations, a clinician uses an interface in a client 106 to enter current cardiovascular performance metrics such left atrial pressure, cardiac output, and mean atrial pressure, as described throughout this disclosure. Alternatively or additionally, the clinician may enter the current cardiovascular parameters. The client 106 may then transmit the entered metrics to the server 102 through the network 104. The drug optimization system 110 may calculate a recommended dosage for the current cardiovascular performance metrics to desired cardiovascular performance metrics (in some embodiments, the clinician may provide the desired cardiovascular performance metrics along with the current cardiovascular performance metrics).

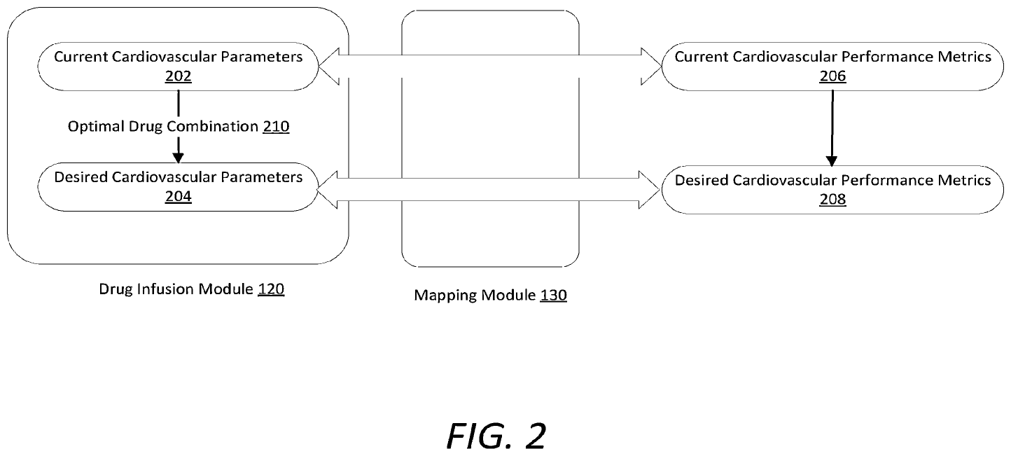

FIG. 2 shows details of the drug infusion module 120 and the mapping module 130, according to example embodiments of this disclosure. It should be understood that the shown details are illustrative and should not be considered limiting. That is, each of the drug infusion module 120 and the mapping module 130 may have other components and/or other processes, which are to be considered within the scope of this disclosure.

As shown, the drug infusion module 120 may include a first analytical model configured to map current cardiovascular parameters 202 to desired cardiovascular parameters 204 by using an optimal drug combination 210. The mapping module 130 may include a second analytical model configured to map the current cardiovascular parameters 202 to the corresponding current cardiovascular performance metrics, and a mapping between the desired cardiovascular parameters 204 and corresponding desired cardiovascular performance metrics 208. While the optimal drug combination 210 may cause measurable changes in the cardiovascular parameters in the drug infusion module 120, the mapping module 130 is configured to allow a clinician to interact with the drug optimization system using the cardiovascular performance metrics.

Embodiments disclosed herein may therefore be based on determining an optimal input of candidate drugs (e.g., optimal drug combination 210) in the drug infusion module 120 such that a desired output (e.g., desired cardiovascular performance metrics 208) may be generated using the mapping module 130. The optimal drug combination 210 may be represented as vector u=[u₁, u₂, u₃, u₄, u₅]^{T}, where each element may correspond to a drug. For example, u₁ may correspond to positive inotropes, u₂ may correspond to vasopressors, u₃ may correspond to vasodilators, u₄ may correspond to fluids, and u₅ may correspond to diuretics. It should, however, be understood that these are just example drugs, which form an example optimal drug combination 210, but should not be considered limiting. Therefore, a generalized input vector u=[u₁, . . . , uₙ]^{T} should be considered within the scope of this disclosure.

In some embodiments, the desired cardiovascular performance metrics 208 may include mean arterial pressure (MAP), left atrial pressure (PLA), and cardiac output (CO). The desired cardiovascular performance metrics may be represented by a desired output vector, y_{d}=[MAP, P_{LA}, CO]^{T}. It should also be understood that these cardiovascular performance metrics are merely examples, and other cardiovascular performance metrics should be considered within the scope of this disclosure. Therefore, similar to the input vector u, the desired output vector y_{d} may also be generalizable to a vector having n elements.

The drug infusion module 120 and the mapping module 130, using the first and second analytical models, may represent a cardiovascular system simulating a mammalian (e.g., human) heart behavior (or cardiovascular behavior). For instance, the drug infusion module 120 and the mapping module 130 may simulate how the heart reacts to a drug and/or a combination of drugs—e.g., the optimal drug combination 210 as indicated by the input vector u=[u₁, . . . uₙ]^{T}.

In some embodiments, the operations of the drug infusion module 120 be linearly represented as x=Bu+X₀, where: x₀ may be an initial state (e.g., current cardiovascular parameters 202) before a drug infusion, u=[u₁, . . . , uₙ]^{T} may be the drug combination (e.g., optimal drug combination 210) that may model impacts of drug infusion to cardiovascular parameters, B may be an interaction matrix (e.g., drug library), and x may be a state (e.g., desired cardiovascular parameters 204) after the drug infusion. As examples of cardiovascular parameters (not to be construed as the only parameters) for x, x₀ states may include systemic vascular resistance (R_{S}), cardiac contractility (Eₑₛ), heart rate (HR), and stressed blood volume (SBV). The desired state (x_{d})—after the drug infusion—may be represented as x_{d}=[R_{S}, Eₑₛ, HR, SBV]^{T}.

The mapping module 130 may be based on hemodynamic analytical model represented by y=h(x), where y is the desired cardiovascular performance metrics 208 output and x is the desired cardiovascular parameters 204 state generated by drug infusion module 120. That is, the s mapping module may map the cardiovascular parameters of a state x to the desired cardiovascular behavior (as indicated by the desired cardiovascular performance metrics 208). For example, optimal drug combination 210 may change the initial state of the cardiovascular parameters (i.e., current cardiovascular parameters 202) to the desired cardiovascular parameters 204 (i.e., x₀ϵX→x_{d}ϵX); thereby changing the mapped current cardiovascular performance metrics 206 to the desired cardiovascular performance metrics 208 (i.e., y₀ϵY→y_{d}ϵY).

In some embodiments, the drug infusion module 120 may model the multiple dependencies of the simultaneous drug infusion when the drug effects have converged. As a five-drug combination example, the input drugs, as detailed above, may be defined as u:=[u₁, . . . , u₅]^{T}∈R⁵. TABLE 1 below shows the example drugs with maximum dosage constraint.

TABLE I
Intervention Drugs for AHF Treatment

| Input | Name | Abb. | Max Dose | Unit |
|---|---|---|---|---|
| u₁ | Dobutamine | DOB | 10 | [μg/kg/min] |
| u₂ | Norepinephrine | NE | 3 | [μg/kg/min] |
| u₃ | Nitroprusside | SNP | 10 | [μg/kg/min] |
| u₄ | Dextran | DEX | 25 | [ml/kg] |
| u₅ | Furosemide | FRO | 180 | [mg] |

As further described above, each of the current cardiovascular parameters 202 and the desired cardiovascular parameters 204 may be defined as x:=[R_{S}, Eₑₛ, HR, SBV]^{T}∈R⁴. Each of the current cardiovascular performance metrics 206 and the desired cardiovascular performance metrics 208 may be defined as y:=[MAP, P_{LA}, CO]^{T}∈R³. As also described above, the drug infusion may be modeled as x=Bu+x₀, y=h(x). In some embodiments, the interaction matrix B may be defined as

$$B = [ \begin{matrix}0 & {k}_{12 +} & {k}_{13 -} & 0 & 0 \\ {k}_{21 +} & {k}_{22 +} & 0 & 0 & 0 \\ 0 & 0 & 0 & 0 & 0 \\ {k}_{41 +} & 0 & {k}_{43 -} & {k}_{44 +} & {k}_{45 -}\end{matrix} ]$$

where kᵢⱼ may indicate the gain: the impact of jth drug impact to ith cardiovascular parameters in steady state.

Using the same symbols, determining the optimal drug combination 210 may be modeled as an optimization problem to minimize an objective function w^{T}u* (i.e., to minimize the weighted dosages) subject to the constraints x_{d}−x₀=Bu* and 0≤u*≤ū, where ū may represent the maximum dosage allowed clinically. The solution of this optimization problem may give the optimal combination u* if the desired state of the cardiovascular parameters x_{d} is given.

However, the desired state of the cardiovascular parameters x_{d} from the desired state of the cardiovascular performance metrics y_{d} may be hard because finding a mapping function (y_{d}ϵY→x_{d}ϵX) is intrinsically difficult due to the inverse nonlinear relationships between the two spaces and their dimensional differences. Embodiments disclosed herein may overcome this problem by solving for X→Y analytically and then finding y_{d}ϵY→x_{d}ϵX numerically.

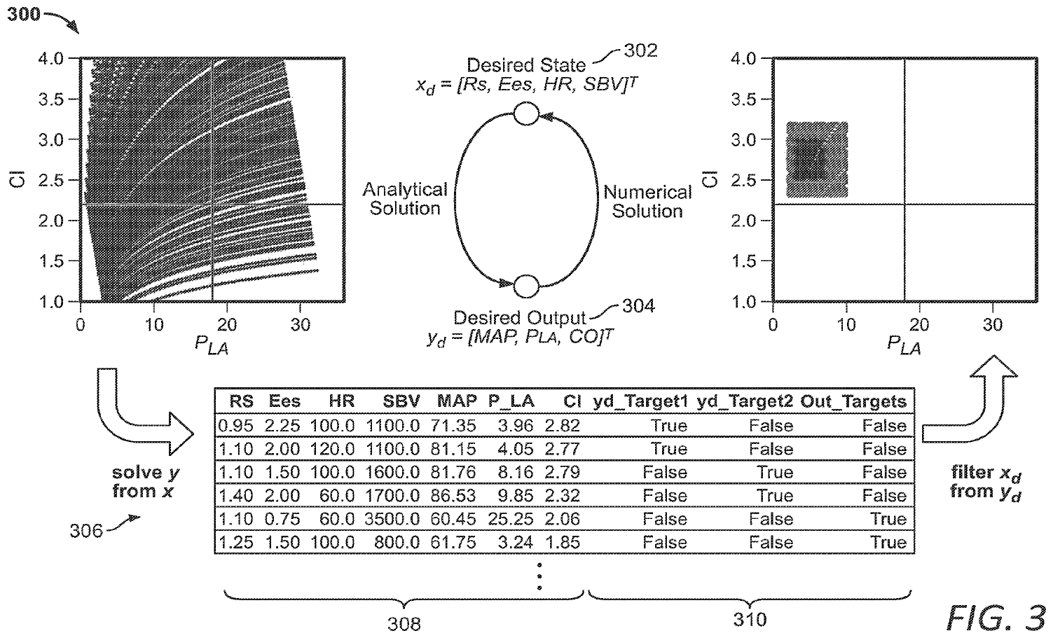

FIG. 3 shows a solution system 300 of the analytical solution followed by the numerical solution of the mapping function utilized by the mapping module 130, according to example embodiments of this disclosure. The analytical solution X→Y from a desired state 302 (i.e., desired cardiovascular parameters 204) to a desired output 304 (i.e., desired cardiovascular performance metrics 208) may be through the use of Frank-Starling Curve and Guyton's Venous Return Curve.

The Frank-Starling Curve may define the relationship between CO and P_{LA} as

$$CO = \frac{HR \cdot {E}_{es}}{β ( {E}_{es} + \frac{HR}{60} \cdot {R}_{s} )} \cdot ln \frac{{P}_{LA} + α}{α} [ \frac{ml}{min} ] \qquad ( 1 )$$

where α and β may be constant parameters to define the end-diastolic pressure volume relationship.

The Guyton's Venous Return Curve may be defined as

$$CO = \frac{1}{{R}_{vp}} ( \frac{{V}_{p}}{{c}_{p}} - {P}_{LA} ) [ \frac{ml}{sec} ] \qquad ( 2 )$$

where Rᵥₚ may be the resistance for pulmonary venous return, and where Cₚ and Vₚ may be the compliance and stressed blood volume in pulmonary circulation.

Assuming that the total stressed blood volume (SBV) is distributed by the compliance ratio of the systemic and pulmonary circulation, Vₚ may be given by:

$${V}_{p} \approx \frac{{C}_{p}}{{C}_{s} + {C}_{p}} SBV \qquad ( 3 )$$

where Cₛ may be the compliance of the systematic circulation. Substituting Vₚ in equation (2) with equation (3) may yield:

$$CO = \frac{1}{{R}_{{v}_{p}}} ( \frac{SBV}{{C}_{p} + {C}_{s}} - {P}_{LA} ) [ \frac{ml}{sec} ] \qquad ( 4 )$$

Solving equation (1) and equation (4) as nonlinear simultaneous equations using Lambert function, the analytical solutions can be obtained as functions of only the cardiovascular parameters as follows:

$${P}_{LA} = - α ( \frac{aW ( - \frac{b}{a} exp ( \frac{c}{a} ) )}{b} ) - α [ mm Hg ]$$

$$\begin{matrix}CO = - aW ( - \frac{b}{a} exp ( \frac{c}{a} ) ) + c [ \frac{L}{min} ] \\ MAP = {R}_{s} CO [ mm Hg ]\end{matrix}$$

where W(.) is defined as a Lambert function and

$$\begin{matrix}a = \frac{1}{1000} \frac{{E}_{es} HR}{β ( {E}_{es} + \frac{HR}{60} {R}_{s} )} \\ b = \frac{60}{1000} \frac{α}{{R}_{vp}} \\ c = \frac{60}{1000} \frac{1}{{R}_{vp}} ( \frac{SBV}{{C}_{s} + {C}_{p}} + α )\end{matrix}$$

The numerical solution may leverage the analytical solution, simulating various patient scenarios (x) and outcomes (y). Particularly, by filtering the Y database within the desired outcome range y_{d}, x_{d}, may be identified. Table 306 shows an example of such filtering. The table 306 shows state parameters 308 (x) and the output parameters 310 (y). The filtering is done to select the output parameters 310 which are in the range, and map the selected output parameters to the state parameters 308.

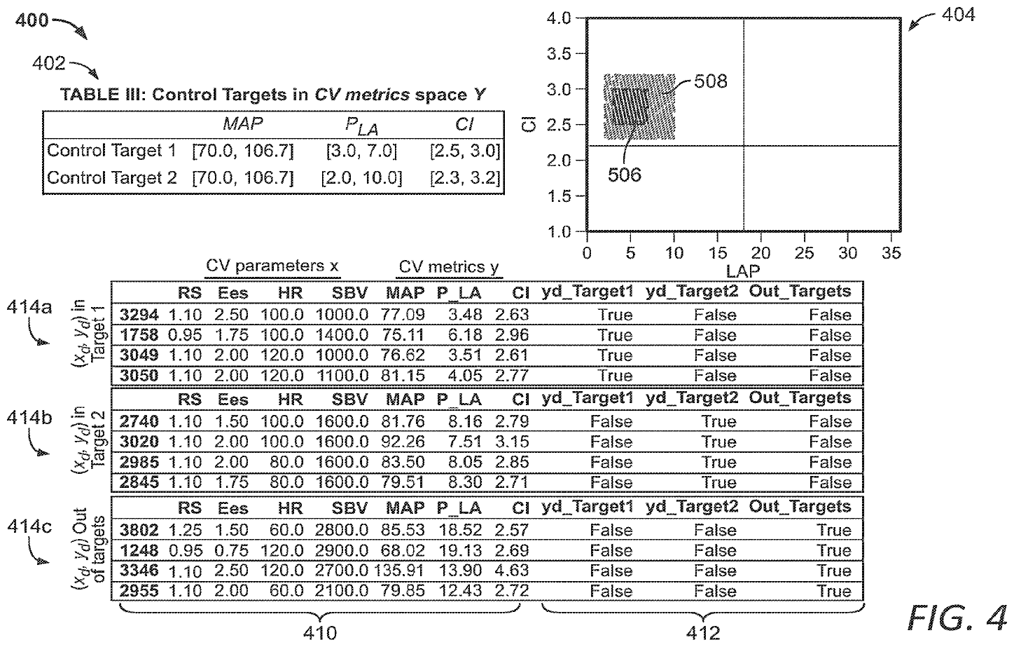

FIG. 4 shows an example solution system 400 of numerical solution from input cardiovascular parameters to output cardiovascular performance metrics utilized by the mapping module 130, according to example embodiments of this disclosure. Table 402 shows the ranges of the control targets of in the cardiovascular performance metrics Y, including MAP, P_{LA}, and Cardiac Index (CI). As shown, there may be two control targets: control target 1 and control target 2. Graph 404 illustrates two-dimensional areas corresponding to the control target 1 and the control target 2. Particularly, area 406 corresponds to the control target 1 and area 408 corresponds to the control target 2. Using the areas 406 and 408, the input cardiovascular parameters x may be filtered out—selecting the cardiovascular parameters that correspond to the control target cardiovascular performance metrics and discarding the rest.

For example, table 414a shows cardiovascular parameters 410 that may generate cardiovascular performance metrics within control target 1, as shown by label 412. Similarly, table 414b shows cardiovascular parameters 410 that generate cardiovascular performance metrics within control target 2, also shown by label 412. Additional, table 414c shows cardiovascular parameters 410 that do not generate cardiovascular performance metrics within either of the control target 1 or the control target 2, as shown by the label 412.

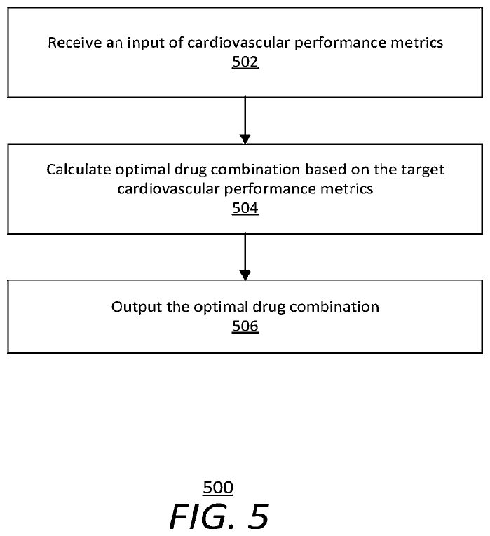

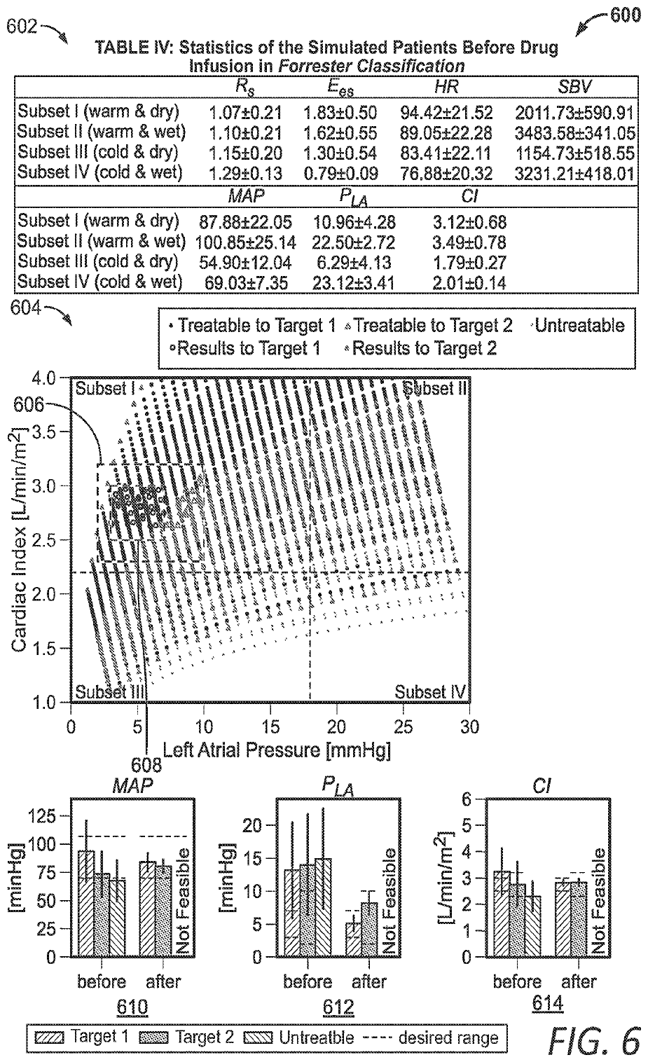

FIG. 5 depicts a flow diagram of an example method 500, based on the example embodiments of this disclosure. The example method 500 may be performed by any combination of components of the computing environment 100 shown in FIG. 1 It should be understood that the steps of the method 500 are just examples and should not be considered limiting. Methods with additional, alternative, or fewer number of steps should be considered within the scope of this disclosure.

The method 500 may begin at step 502. At step 502, server 102 may receive an input of cardiovascular performance metrics. For example, a desktop terminal in a hospital terminal may be used by a clinician to enter the cardiovascular performance metrics. Alternatively, the clinician may enter the cardiovascular performance metrics on a smartphone or a tablet computer. The cardiovascular performance metrics may include, for example, current cardiovascular performance metrics and/or target cardiovascular performance metrics. It should be understood that the clinician may enter current cardiovascular parameters in lieu of or in addition to the current cardiovascular performance metrics.

At step 504, server 102 may calculate an optimal drug combination based on the target cardiovascular performance metrics. In some embodiments, regardless of the modality of the entry of the desired cardiovascular performance metrics, the server 102 may be execute step 504 to calculate the optimal drug combination based on the target cardiovascular performance metrics. For example, the drug optimization system 110 may deploy the drug infusion module 120 to determine an optimal drug combination to reach target cardiovascular metrics from the current cardiovascular metrics. The drug optimization system 110 may deploy the mapping module 130 to map the current cardiovascular parameters to the current cardiovascular performance metrics, and the target cardiovascular parameters to the target cardiovascular performance metrics.

At step 506, server 102 may output the optimal drug combination (e.g., at the requesting device) to assist clinical decision making. That is, the clinician can rely on the tested and simulated models to aid the decision making and rely less on guesswork. In some embodiments, the server 102 may not necessarily calculate the optimal drug calculation, e.g., the described analytical models may be out of range for a specific patient. In these cases, the server 102 may output an indication that the patient is untreatable.

FIG. 6 shows an example system 600 for treatability simulation, according to example embodiments of this disclosure. The treatability simulation may be performed by the drug optimization system 110 shown in FIG. 1 For the treatability simulation, constant parameters may be picked. Table II below shows example parameters.

TABLE II
Constant Parameters used in Experiments

| Const. | Value | Unit (γ = μg/kg/min) | Description |
|---|---|---|---|
| k₁₂₊ | 0.100 | [(mmHg · s)/(γ · ml)] | NE → Rₛ |
| k₁₃₋ | −0.211 | [ml/(γ · kg)] | SNP → Rₛ |
| k₂₁₊ | 0.0625 | [(mmHg)/(γ · ml)] | DOB → Eₑₛ |
| k₂₂₊ | 0.050 | [(mmHg)/(γ · ml)] | NE → Eₑₛ |
| k₄₁₊ | 0.339 | [ml/(γ · kg)] | DOB → SBV |
| k₄₃₋ | −1.430 | [ml/(γ · kg)] | SNP → SBV |
| k₄₄₊ | 1.000 | [ml/ml] | DEX → SBV |
| k₄₅ | −0.400 | [ml/(mg · kg)] | FRO → SBV |
| α | 0.440 | unitless | EDPVR |
| β | 0.030 | unitless | EDPVR |
| Cₚ | 18.6 | [ml/mmHg] | Pulmonary Compliance |
| Cₛ | 102.0 | [ml/mmHg] | Systemic Compliance |
| Rᵥₚ | 0.0686 | [mmHg · sec/ml] | Pulmonary VR Resistance |
| ω_{1...4} | 1.0 | unitless | weights ω in (2) |
| ω₅ | 0.1 | unitless | weight ω in (2) |

The patients may be categorized into four subsets according tom Forrester Classification: Subset I (warm and dry), Subset II (warm and wet), Subset III (cold and dry), and Subset IV (cold and wet). Table 602 shows statistics (both cardiovascular parameters and cardiovascular performance metrics) for the patients before infusion the drugs. In the table 602, cardiac index (CI) is defined as CO/body surface area (BSA). The BSA herein may be set at an average value of 1.6 m².

The treatability analysis may be used to validate the proposed optimization system. Two control targets were chosen based on the table 402 shown in FIG. 4. Based on the choice of the control targets, the treatability analysis may generate three results: (1) treatable to target 1: if the optimization solution is feasible for at least one of the randomly selected patients in the control target 1; (2) treatable to target 2: if a patient is not treatable to target 1 and the optimization solution is feasible to at least one of the randomly selected patients in the control target 2; and (3) untreatable: if the patient is not treatable to either target 1 or target 2. Graph 604 shows the result for two cardiovascular performance metrics P_{LA} and CI. Within the graph 604, results to target 1 are shown as 608 and results to target 2 are shown as 606. Graph 610 shows the before and after MAP statistics for each of the target 1 patients, target 2 patients, and untreatable patients. Graph 612 shows before and after P_{LA} statistics for each of the target 1 patients, target 2 patients, and untreatable patients. Graph 614 shows before and after CI statistics for each of the target 1 patients, target 2 patients, and untreatable patients. Therefore, embodiments disclosed herein may also generate whether a cohort of patients is treatable or whether alternate non-drug (e.g., mechanical types) of treatment may have to be devised.

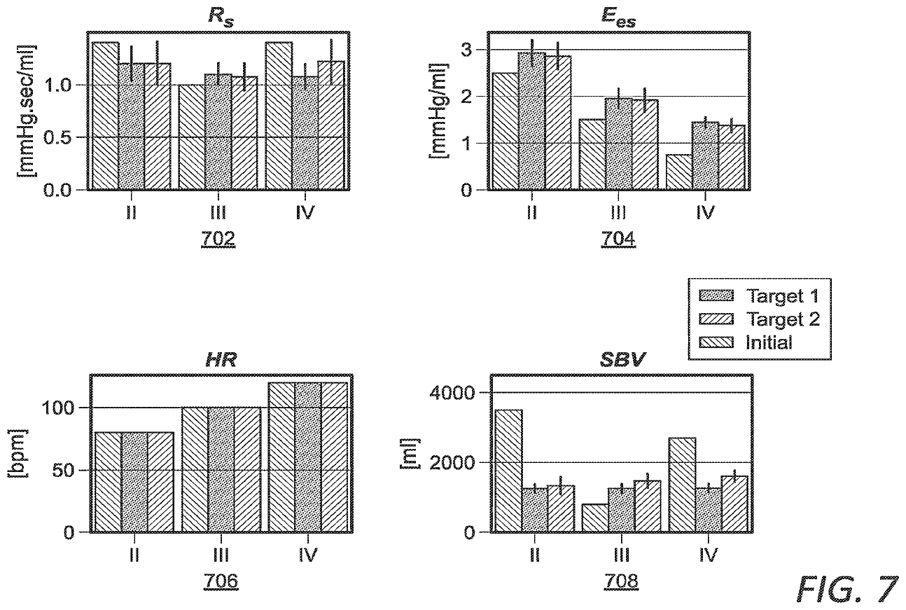

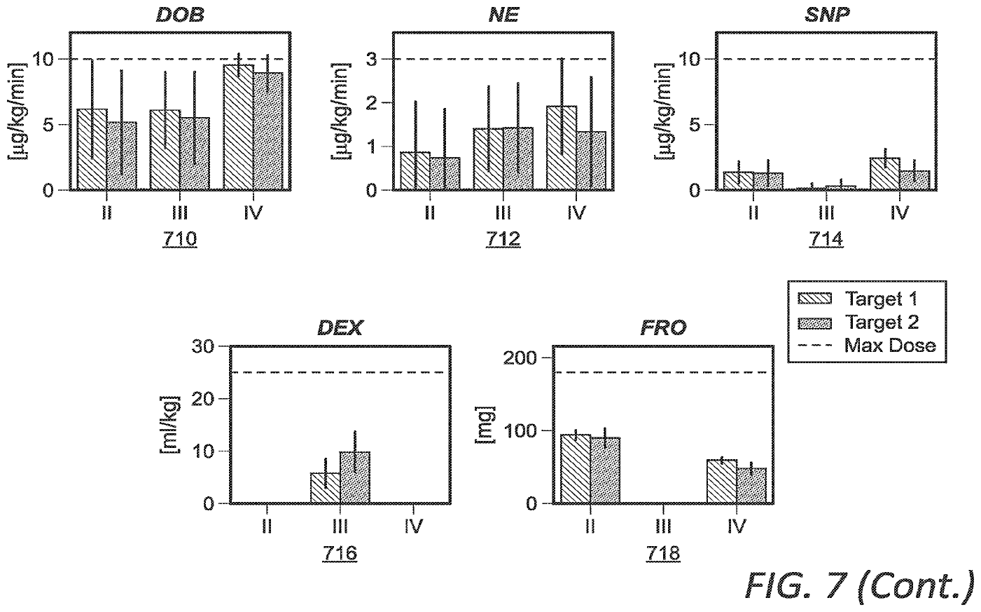

FIG. 7 shows example graphs 702-718 for a pathophysiological scenario analysis, according to example embodiments of this disclosure. The pathophysiological scenario analysis may be performed by the drug optimization system 110 shown in FIG. 1. The pathophysiological scenario analysis may for a three patient scenario: (1) warm and wet patient in Subset II,: [R_{S}=1.4, Eₑₛ=2.5, HR=80, SBV=3500]; (2) cold and dry patient in Subset III,: [=1.0, =1.5, HR=100, SBV=800]; and (3) cold and wet patient in Subset IV,: [R_{S}=1.4, Eₑₛ=0.75, HR=120, SBV=2700]. Each of the graphs 702 (for cardiovascular parameter R_{S}), 704 (for cardiovascular parameter Eₑₛ), 706 (for cardiovascular parameter HR), and 708 (for cardiovascular parameter SBV) show initial state, target 1 state, and target 2 state for the three patients. Graphs 710, 712, 714, 716, and 718 show tailored (or optimized) drug infusion (target 1, target 2, and maximum dosage) for each patient in the Subsets II, III, and IV. Particularly, graph 710 shows optimized amounts of dobutamine (DOB), graph 712 shows optimized amounts of norepinephrine (NE), graph 714 shows optimized amounts of sodium nitroprusside (SNP), graph 716 shows optimized amounts of dextran (DEX), and graph 718 shows optimized amounts of furosemide (FRO). As shown, the optimized drugs are under the maximum dosages and within the recommended clinical use guidelines.

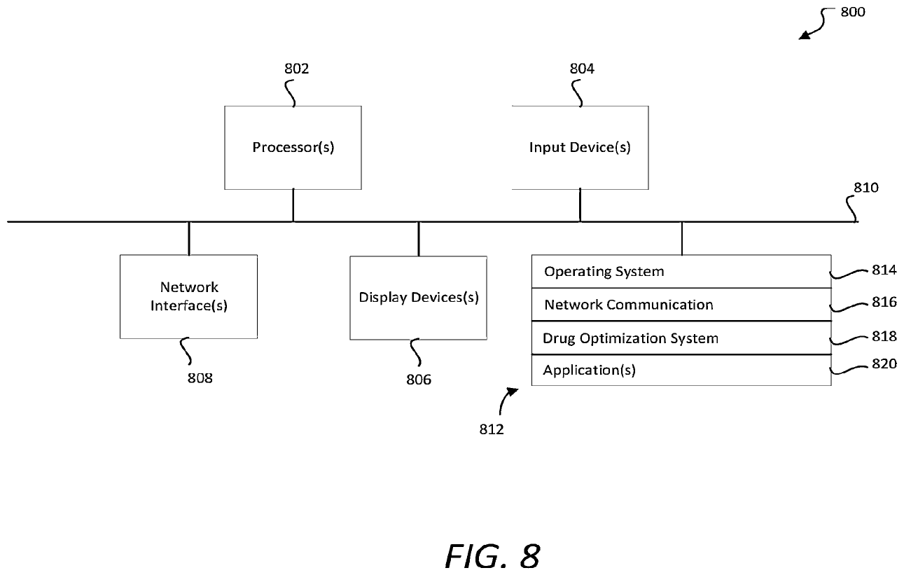

FIG. 8 shows a block diagram of an example computing device 800 that implements various features and processes, according to example embodiments of this disclosure. For example, computing device 800 may function as the server 102 and clients 106, or a portion or combination thereof in some embodiments. The computing device 800 may also perform one or more steps of the methods 500. The computing device 800 is implemented on any electronic device that runs software applications derived from compiled instructions, including without limitation personal computers, servers, smart phones, media players, electronic tablets, game consoles, email devices, etc. In some implementations, the computing device 800 includes one or more processors 802, one or more input devices 804, one or more display devices 806, one or more network interfaces 808, and one or more computer-readable media 812. Each of these components is be coupled by a bus 810.

Display device 806 includes any display technology, including but not limited to display devices using Liquid Crystal Display (LCD) or Light Emitting Diode (LED) technology. Processor(s) 802 uses any processor technology, including but not limited to graphics processors and multi-core processors. Input device 804 includes any known input device technology, including but not limited to a keyboard (including a virtual keyboard), mouse, track ball, and touch-sensitive pad or display. Bus 810 includes any internal or external bus technology, including but not limited to ISA, EISA, PCI, PCI Express, USB, Serial ATA or FireWire. Computer-readable medium 812 includes any non-transitory computer readable medium that provides instructions to processor(s) 802 for execution, including without limitation, non-volatile storage media (e.g., optical disks, magnetic disks, flash drives, etc.), or volatile media (e.g., SDRAM, ROM, etc.).

Computer-readable medium 812 includes various instructions 814 for implementing an operating system (e.g., Mac OS®, Windows®, Linux). The operating system may be multi-user, multiprocessing, multitasking, multithreading, real-time, and the like. The operating system performs basic tasks, including but not limited to: recognizing input from input device 804; sending output to display device 806; keeping track of files and directories on computer-readable medium 812; controlling peripheral devices (e.g., disk drives, printers, etc.) which can be controlled directly or through an I/O controller; and managing traffic on bus 810. Network communications instructions 816 establish and maintain network connections (e.g., software for implementing communication protocols, such as TCP/IP, HTTP, Ethernet, telephony, etc.).

Drug optimization system 818 includes instructions that implement the disclosed process for determining optimal combination of drugs for heart failure patients, as described throughout this disclosure. Application(s) 820 may comprise an application that uses or implements the processes described herein and/or other processes. The processes may also be implemented in the operating system.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment. In one embodiment, this may include Python. The computer programs therefore are polyglots.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

Additional examples of the presently described method and device embodiments are suggested according to the structures and techniques described herein. Other non-limiting examples may be configured to operate separately or can be combined in any permutation or combination with any one or more of the other examples provided above or throughout the present disclosure.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It should be noted that the terms “including” and “comprising” should be interpreted as meaning “including, but not limited to”. If not already set forth explicitly in the claims, the term “a” should be interpreted as “at least one” and “the”, “said”, etc. should be interpreted as “the at least one”, “said at least one”, etc. Furthermore, it is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A computer readable non-transitory storage medium storing computer program instructions, that when executed cause operations comprising:
receiving a plurality of current cardiovascular performance metrics of a patient and a plurality of candidate drugs to be used to reach a plurality of desired cardiovascular performance metrics;
determining optimal dosages of the plurality of candidate drugs to reach the plurality of desired cardiovascular performance metrics, the determining comprising:
optimizing a dosage combination of the plurality of candidate drugs to reach a plurality of desired cardiovascular parameters, corresponding to the plurality of desired cardiovascular performance metrics, from a plurality of current cardiovascular parameters corresponding to the plurality of current cardiovascular performance metrics; and
mapping the plurality of desired cardiovascular performance metrics from the plurality of desired cardiovascular parameters and the plurality of current cardiovascular performance metrics from the plurality of current cardiovascular parameters; and
outputting the optimal dosages of the plurality of candidate drugs.

2. The computer readable non-transitory storage medium of claim 1, wherein the plurality of current cardiovascular performance metrics and the plurality of desired cardiovascular performance metrics comprise one or more of mean arterial pressure, left atrial pressure, cardiac output, or cardiac index.

3. The computer readable non-transitory storage medium of claim 1, wherein the plurality of current cardiovascular parameters and the plurality of desired cardiovascular parameters comprise one or more of systemic vascular resistance, cardiac contractility, heart rate, or stressed blood volume.

4. The computer readable non-transitory storage medium of claim 1, wherein the plurality of candidate drugs comprises at least one of positive inotropes, vasopressors, vasodilators, fluids, or diuretics.

5. The computer readable non-transitory storage medium of claim 1, wherein the optimal dosages of the plurality of candidate drugs is constrained by a linear relationship between the plurality of candidate drugs, the plurality of current cardiovascular parameters, and the plurality of desired cardiovascular parameters.

6. The computer readable non-transitory storage medium of claim 1, wherein the optimal dosages of the plurality of candidate drugs is constrained by a maximum dosage limitation of each of the plurality of candidate drugs.

7. The computer readable non-transitory storage medium of claim 1, wherein the mapping of the plurality of desired cardiovascular performance metrics from the plurality of desired cardiovascular parameters is based on a hemodynamics analysis.

8. The computer readable non-transitory storage medium of claim 7, wherein the hemodynamics analysis comprises analytically deriving the plurality of desired cardiovascular performance metrics from the plurality of desired cardiovascular parameters.

9. The computer readable non-transitory storage medium of claim 7, wherein the hemodynamics analysis comprises numerically determining the plurality of desired cardiovascular parameters based on a filtering using the plurality of desired cardiovascular performance metrics.

10. The computer readable non-transitory storage medium of claim 1, wherein outputting the optimal dosages of the plurality of candidate drugs comprises:
displaying the optimal dosages on a screen.

11. A computer-implemented method comprising:
receiving a plurality of current cardiovascular performance metrics of a patient and a plurality of candidate drugs to be used to reach a plurality of desired cardiovascular performance metrics;
determining optimal dosages of the plurality of candidate drugs to reach the plurality of desired cardiovascular performance metrics, the determining comprising:
optimizing a dosage combination of the plurality of candidate drugs to reach a plurality of desired cardiovascular parameters, corresponding to the plurality of desired cardiovascular performance metrics, from a plurality of current cardiovascular parameters corresponding to the plurality of current cardiovascular performance metrics; and
mapping the plurality of desired cardiovascular performance metrics from the plurality of desired cardiovascular parameters and the plurality of current cardiovascular performance metrics from the plurality of current cardiovascular parameters; and
outputting the optimal dosages of the plurality of candidate drugs.

12. The computer-implemented method of claim 11, wherein the plurality of current cardiovascular performance metrics and the plurality of desired cardiovascular performance metrics comprise one or more of mean arterial pressure, left atrial pressure, cardiac output, or cardiac index.

13. The computer-implemented method of claim 11, wherein the plurality of current cardiovascular parameters and the plurality of desired cardiovascular parameters comprises one or more of systemic vascular resistance, cardiac contractility, heart rate, or stressed blood volume.

14. The computer-implemented method of claim 11, wherein the plurality of candidate drugs comprises at least one of positive inotropes, vasopressors, vasodilators, fluids, or diuretics.

15. The computer-implemented method of claim 11, wherein the optimal dosages of the plurality of candidate drugs is constrained by a linear relationship between the plurality of candidate drugs, the plurality of current cardiovascular parameters, and the plurality of desired cardiovascular parameters.

16. The computer-implemented method of claim 11, wherein the optimal dosages of the plurality of candidate drugs is constrained by a maximum dosage limitation of each of the plurality of candidate drugs.

17. The computer-implemented method of claim 11,, wherein the mapping of the plurality of desired cardiovascular performance metrics from the plurality of desired cardiovascular parameters is based on a hemodynamics analysis.

18. The computer-implemented method of claim 17, wherein the hemodynamics analysis comprises analytically deriving the plurality of desired cardiovascular performance metrics from the plurality of desired cardiovascular parameters.

19. The computer-implemented method of claim 17, wherein the hemodynamics analysis comprises numerically determining the plurality of desired cardiovascular parameters based on a filtering using the plurality of desired cardiovascular performance metrics.

20. A system comprising:
a non-transitory computer readable medium storing computer program instructions; and
one or more processors configured to execute the computer program instructions to cause operations comprising:
receiving a plurality of current cardiovascular performance metrics of a patient and a plurality of candidate drugs to be used to reach a plurality of desired cardiovascular performance metrics;
determining optimal dosages of the plurality of candidate drugs to reach the plurality of desired cardiovascular performance metrics, the determining comprising:
optimizing a dosage combination of the plurality of candidate drugs to reach a plurality of desired cardiovascular parameters, corresponding to the plurality of desired cardiovascular performance metrics, from a plurality of current cardiovascular parameters corresponding to the plurality of current cardiovascular performance metrics; and
mapping the plurality of desired cardiovascular performance metrics from the plurality of desired cardiovascular parameters and the plurality of current cardiovascular performance metrics from the plurality of current cardiovascular parameters; and
outputting the optimal dosages of the plurality of candidate drugs.
